(19) United States
(12) Patent Application Publication
(10) Pub. No.: US 2005/0027288 A1
(43) Pub. Date: Feb. 3, 2005

(54) Laser operation apparatus

(75) Inventors: Wataru Oyagi, Tokyo (JP); Kazuhisa Nomura, Tokyo (JP); Iori Nemoto, Tokyo (JP); Hideo Sagehashi, Tokyo (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: KABUSHIKI KAISHA TOPCON, Tokyo (JP)

(21) Appl. No.: 10/896,047

(22) Filed: Jul. 22, 2004

(30) Foreign Application Priority Data
Jul. 31, 2003 (JP) 2003-204428

(51) Int. Cl. A61B 18/18

(52) U.S. Cl. 606/16; 606/2

(57) ABSTRACT

There is provided a laser operation apparatus capable of emitting suitable laser light having a uniformed light intensity distribution. A fiber vibrating device is fit to a portion of an optical fiber that guides laser light oscillated (for treatment) by a treatment laser light source to a slit lamp. The fiber vibrating device itself is vibrated by rotating a weight provided eccentric about a rotational shaft by a motor, thereby vibrating the optical fiber in a direction substantially orthogonal to an axial line direction thereof. When the optical fiber is vibrated by the fiber vibrating device, the laser light guided through the optical fiber is mixed to uniform the light intensity distribution of the laser light. The laser light having the uniformed light intensity distribution is emitted to an eye to be operated by the slit lamp.

### BACKGROUND OF THE INVENTION

1. Field of the Invention

The present invention relates to a laser operation apparatus for irradiating a treatment site with laser light to conduct an operation. More particularly, the present invention relates to a laser operation apparatus capable of irradiating a treatment site with suitable laser light having a uniform light intensity distribution.

2. Description of the Related Art

In recent years, treatment site operations using laser light, including a photo-coagulation, a removal, and an incision have been actively conducted. According to such an operation manner, a non-contact treatment can be conducted on the treatment site. Therefore, even when it is improper to directly touch the treatment site as in, for example, an ophthalmologic operation, a suitable treatment can be conducted. In addition, the operation manner has many advantages in that the treatment site can be prevented from bleeding and a fear of contamination from bacteria is extremely low. Thus, for the future, it has been expected that the operation manner becomes more popular.

A laser operation apparatus used for those operations is generally divided into “a parfocal type same focus” and “a defocus type off-set focus” according to a focusing mode.

When a step index optical fiber is used in the parfocal type laser operation apparatus, an exit end surface of the optical fiber and the treatment site are optically conjugate with each other. Therefore, the treatment site is spotted with laser light while maintaining a light intensity distribution in a fiber near field. At this time, the light intensity distribution of the laser light becomes a rectangular form in which edges are sharp.

On the other hand, in the case of the defocus type laser operation apparatus, the exit end surface of the optical fiber and the treatment site are not conjugate with each other. Therefore, the laser light with which the treatment site is spotted has a light intensity distribution in which edges are not sharp and a light strength in a central region is relatively high.

In general, it is considered that laser light having a rectangular light intensity distribution in which edges are sharp or laser light having a light intensity distribution in which a light strength in a central region is low is preferable for a photo-coagulation treatment on a retina.

An apparatus disclosed in JP 2001-008945 A will be briefly described as an example of the laser operation apparatus. The laser operation apparatus has an external structure shown in FIG. 9.

The laser operation apparatus includes: an apparatus main body 100 containing a laser light source (described later) for oscillating laser light; a control panel 200 for performing various operations and settings of the apparatus main body 100; a slit lamp 400 placed on a table 300; a foot switch 500 for performing various operations of the apparatus main body 100 and the slit lamp 400; and an optical fiber 600 for guiding the laser light oscillated by the apparatus main body 100 to the slit lamp 400.

The spit lamp 400 is provided with a control lever 401 used for an alignment with a patients eye and an operation for irradiating the patient's eye with the laser light and an irradiation optical system unit 402 containing an irradiation optical system for irradiating the patient's eye with the laser light. Note that an irradiation switch which is not shown is provided at an upper portion of the control lever 401. The laser light irradiation is performed by pressing down the irradiation switch.

When the foot switch 500 is connected with the apparatus main body 100, the laser light irradiating operation can be conducted by the foot switch 500 instead of the irradiation switch.

The apparatus main body 100 contains a treatment laser light source and a sighting laser light source. The treatment laser light source is a laser light source for oscillating treatment laser light with which the treatment site is irradiated for a photo-coagulation or the like. The sighting laser light source is a laser light source for oscillating sighting laser light for aligning an irradiation position of the treatment laser light with the treatment site. The laser light oscillated by each of the laser light sources is condensed to an incident end surface of the optical fiber 600 by a condensing lens (not shown) in the apparatus main body 100. The condensed laser light is guided to the irradiation optical system unit 402 of the slit lamp 400 through the optical fiber 600 to irradiate an interior of the eye.

The irradiation optical system unit 402 of the slit lamp 400 is provided with a lens group for changing a focusing position of the laser light. When the lens group is moved in an optical axis direction, a spot size of the laser light on the treatment site can be adjusted.

The laser operation apparatus described in JP 2001-008945 A is further provided with a light intensity changing unit for changing a light intensity distribution of the spotlight. The light intensity changing unit includes a circular filter holding plate rotated by a motor. A plurality of filters having different characteristics are arranged on the filter holding plate. One of the filters on the filter holding plate is selectively aligned to the exit end surface of the optical fiber 600 by the rotation of the motor. Therefore, it is possible to generate spot light having one of various light intensity distributions corresponding to the characteristics of the respective filters.

The light intensity distribution in the spot of laser light (laser spot) greatly depends on not only the focusing mode but also the property of a laser light source. A laser light source having a preferred property is generally expensive, so that a laser operation apparatus including such a laser light source becomes relatively expensive. Therefore, even in the parfocal type laser operation apparatus using the step index optical fiber, the light intensity distribution is not uniformed in some cases. Thus, there is an aspect that the accuracy of the operation is maintained by the operator's skill.

When the light intensity distribution of laser light transmitted through a filter is not uniformed in the conventional laser operation apparatus, laser light accurately reflecting the characteristic of the filter cannot be generated.

In order to uniform the light intensity distribution of laser light, for example, a method of using a diffusion plate is devised. It is undeniable that a practical problem occurs in that a loss of the laser light increases.

### SUMMARY OF THE INVENTION

The present invention has been made in view of the above-mentioned circumstances. An object of the present invention is to provide a laser operation apparatus capable of emitting suitable laser light having a uniformed light intensity distribution.

To attain the above object, according to a first aspect of the present invention, a laser operation apparatus includes: a laser oscillator; an optical fiber for guiding laser light oscillated by the laser oscillator; an irradiation optical system for irradiating a treatment site of a patient with the laser light guided through the optical fiber during an alignment with the treatment site; and vibrating means for vibrating a portion of the optical fiber in a direction substantially orthogonal to a longitudinal direction of the optical fiber.

Further, to attain the above object, according to a second aspect of the present invention, in the laser operation apparatus according to the first aspect, the vibrating means is controlled so as to start and stop a vibration of the optical fiber in accordance with a timing of irradiation of the treatment site with the laser light from the irradiation optical system.

Further, to attain the above object, according to a third aspect of the present invention, the laser operation apparatus according to the second aspect further includes an irradiation switch operated to start the irradiation of the treatment site with the laser light from the irradiation optical system, wherein the vibrating means is controlled so as to start the vibration of the optical fiber in accordance with an operation of the irradiation switch.

Further, to attain the above object, according to a fourth aspect of the present invention, the laser operation apparatus according to the third aspect further includes a ready switch operated before the operation of the irradiation switch in order to enable the irradiation switch, wherein the vibrating means is controlled so as to start the vibration of the optical fiber in accordance with an operation of the ready switch.

### BRIEF DESCRIPTION OF THE DRAWINGS

In the accompanying drawings:

FIG. 1 is a schematic view showing an example of a structure of an optical system included in a laser operation apparatus according to an embodiment of the present invention;

FIGS. 2A and 2B are schematic views showing an example of a structure of a fiber vibrating device included in the laser operation apparatus according to the embodiment of the present invention;

FIG. 3A shows an example of a fitting configuration of the fiber vibrating device in the laser operation apparatus according to the embodiment of the present invention, which shows an example of a fitting configuration in which an optical fiber is inserted into a case of the fiber vibrating device, and FIG. 3B shows an example of a fitting configuration in which the optical fiber is fixed to an external surface of the case of the fiber vibrating device;

FIG. 4A is a graph showing a light intensity distribution of laser light at an exit end surface of the optical fiber in the laser operation apparatus according to the embodiment of the present invention, and FIG. 4B is a graph showing a light intensity distribution of laser light emitted to a treatment site;

FIG. 5A is a graph for explaining an operation and an effect of the laser operation apparatus according to the embodiment of the present invention, showing a light intensity distribution of laser light emitted to the treatment site when the optical fiber is not vibrated, and FIG. 5B is a graph showing a light intensity distribution of laser light emitted to the treatment site when the optical fiber is vibrated;

FIG. 6 is a schematic block diagram showing a structure of a modified example of the laser operation apparatus according to the embodiment of the present invention;

FIG. 7 is a schematic block diagram showing a structure of a modified example of the laser operation apparatus according to the embodiment of the present invention;

FIG. 8 is a schematic view showing a structure of an optical system in a modified example of the laser operation apparatus according to the embodiment of the present invention; and

FIG. 9 is a schematic view showing an external structure of a conventional laser operation apparatus.

### DETAILED DESCRIPTION OF THE PREFERRED EMBODIMENT

Hereinafter, an example of a laser operation apparatus according to an embodiment of the present invention will be described in detail with reference to the drawings. The same reference symbols are used for the same structural parts as in the conventional laser operation apparatus.

A laser operation apparatus according to this embodiment is used to operate a treatment site of a patient's eye (which is called an eye to be operated) by a photo-coagulation, which is a parfocal type laser operation apparatus using a step index optical fiber as an optical fiber 600.

[External Structure]

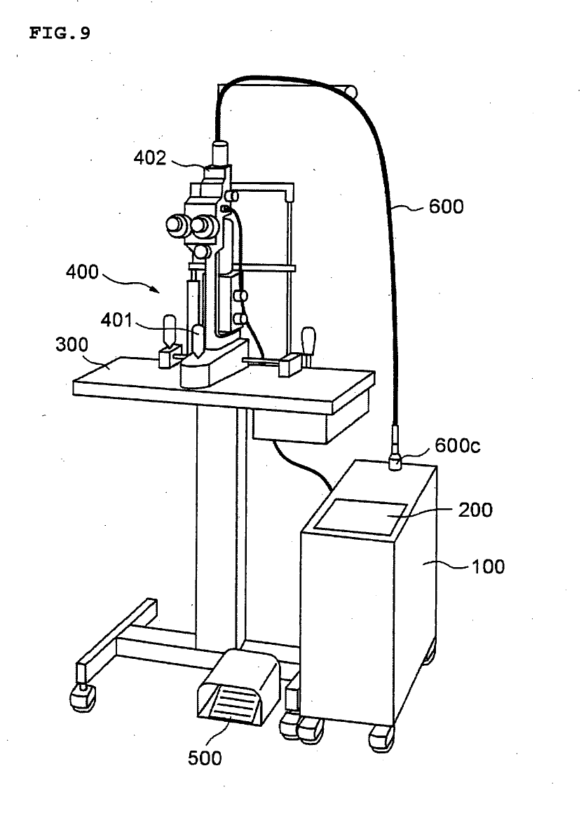

The laser operation apparatus according to this embodiment has substantially the same external structure as the conventional one (see FIG. 9). Note that the laser operation apparatus according to this embodiment is different from the conventional one in a point that a fiber vibrating device for vibrating the optical fiber 600 as described later is externally provided.

[Optical Structure]

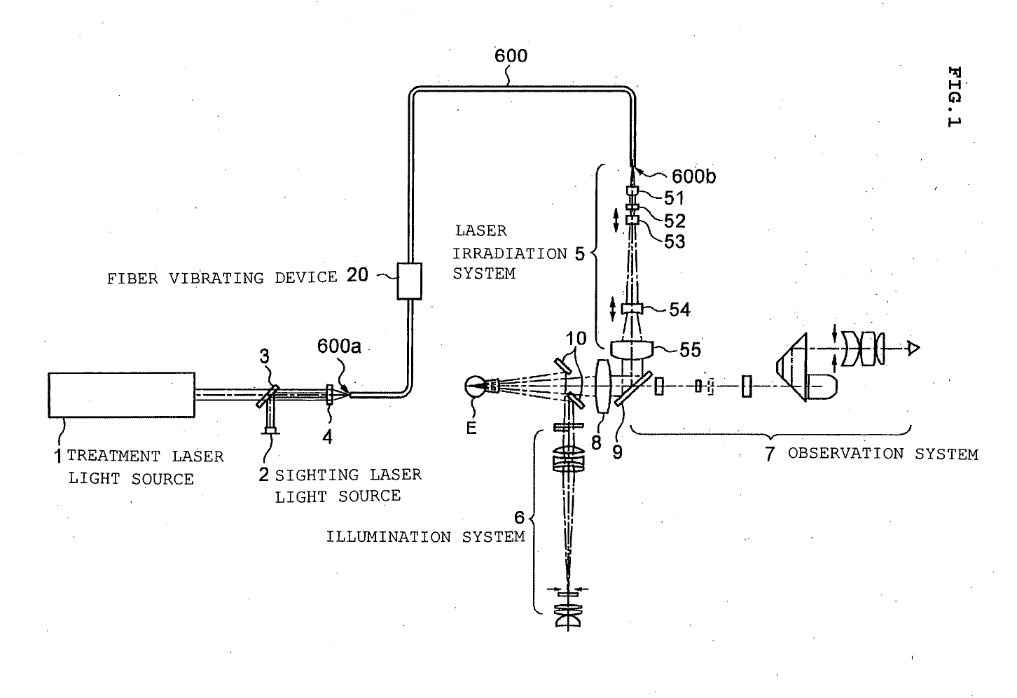

FIG. 1 shows structures of various optical system included in the laser operation apparatus according to this embodiment. The laser operation apparatus includes a treatment laser light source 1 and a sighting laser light source 2 corresponding to a laser oscillating unit in the present invention. The treatment laser light source 1 oscillates treatment laser light for optically coagulating a treatment site on a retina or the like of an eye to be operated E. The sighting laser light source 2 oscillates sighting laser light for aligning an irradiation position of the treatment laser light with the treatment site. The treatment laser light source 1 and the sighting laser light source 2 are contained in the apparatus main body 100.

An optical path of the treatment laser light and an optical path of the sighting laser light are combined with each other by a dichroic mirror 3. Synthesized laser light travels through the dichroic mirror 3, and then is condensed to an incident end surface 600a of the optical fiber 600 by a condensing lens 4 and guided to an irradiation optical system unit 402 of a slit lamp 400 through the optical fiber 600.

A known laser irradiation system 5 is provided as an irradiation optical system in the present invention in the irradiation optical system unit 402 of the slit lamp 400. The laser irradiation system 5 includes: a lens 51 for converting laser light exited from an exit end surface 600b of the optical fiber 600 into a parallel light flux; a lens 52 for condensing the parallel light flux converted by the lens 52; lenses 53 and 54 for changing a focusing position of the laser light condensed by the lens 52 on an eye to be operated E; and a lens 55 for converting the laser light transmitted through the lenses 53 and 54 into a parallel light flux again. The lenses 53 and 54 are moved in an optical axis direction by a cam mechanism which is not shown, thereby changing the focusing position of the laser light on the eye to be operated E.

Although not described in detail, the slip lamp 400 contain a known illumination system 6 for irradiating the eye to be operated E with illumination light and a known observation system 7 for observing the eye to be operated E. The illumination system 6 includes a light source located below due to an inner space of the slit lamp 400. The observation system 7 includes a pair of right and left optical systems capable of binocularly viewing an observation image of the eye to be operated E by an operator. An image pickup element for taking the observation image is provided in the observation system 7 in some cases.

The slit lamp 400 further includes an objective lens 8, a reflecting mirror 9, and a split illumination mirror 10. The objective lens 8 acts to focus laser light transmitted through the laser irradiation system 5 and the reflecting mirror 9 to the interior of the eye to be operated E and to guide a reflection light flux of the laser light to the observation system 7. The reflecting mirror 9 is obliquely provided with respect to the optical axis of the laser irradiation system 5 and the optical axis of the observation system 7 to reflect the laser light guided to the laser irradiation system 5 to the eye to be operated E. The reflecting mirror 9 has a transmitting portion for transmitting the observation light from the eye to be operated E, which is not shown. The observation light is incident on the observation system 7 through the transmitting portion. The split illumination mirror 10 is an optical member for reflecting the illumination light from the illumination system 6 to the eye to be operated E, which is disposed at such a position that the laser light emitted to the eye to be operated E and a reflection light thereof are not blocked.

[Structure of Fiber Vibrating Device]

A fiber vibrating device 20 for vibrating the optical fiber 600 is provided in the laser operation apparatus according to this embodiment. As shown in FIG. 1, the fiber vibrating device 20 is connected with the optical fiber 600 and vibrates a connection portion therewith and the vicinity thereof in a direction substantially orthogonal to the axial direction (longitudinal direction) of the optical fiber 600. Note that the fiber vibrating device 20 composes a vibrating means in the present invention.

The fiber vibrating device 20 can be connected with the optical fiber 600 at an arbitrary position. However, it is improper to dispose the fiber vibrating device 20 near the incident end surface 600a and the exit end surface 600b of the optical fiber 600. That is, because of a loss of the laser light, high precision is required to condense laser light to the incident end surface 600a by the condensing lens 4. Therefore, it is improper to generate a vibration by which the position of the incident end surface 600a is displaced. When a vibration by which the position of the exit end surface 600b is displaced is applied, a relative position between the lens 51 of the laser irradiation system 5 and the exit end surface 600b varies to cause the loss of laser light. Therefore, such a vibration is also improper. As a result, the fiber vibrating device 20 may be connected with the optical fiber 600 at any position as long as the incident end surface 600a and the exit end surface 600b are not vibrated.

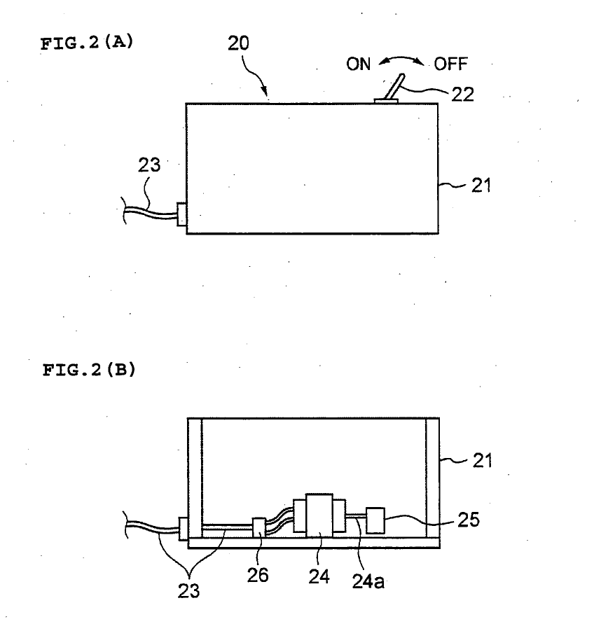

FIGS. 2A and 2B show a constructional example of the fiber vibrating device 20. FIG. 2A is a schematic view showing an external structure of the fiber vibrating device 20 and FIG. 2B is a schematic view showing an internal structure thereof.

As shown in FIG. 2A, the fiber vibrating device 20 includes a case 21, a switch 22 provided on the case 21, and a power source cord 23. The fiber vibrating device 20 is turned ON/OFF by switching operation of the switch 22 serving as a changing switch in the present invention.

The case 21 of the fiber vibrating device 20 houses a motor 24, a weight 25, and a connection member 26. The weight 25 is provided eccentric about a rotational shaft 24a of the motor 24. The power source cord 23 and the motor 24 are connected with each other through the connection member 26. The connection member 26 switches between ON/OFF states of power supply to the motor 24 according to the ON/OFF of the switch 22.

When the switch 22 of the fiber vibrating device 20 is turned ON, a state of power supply to the motor 24 is switched to the ON state by the connection member 26, so that the motor 24 rotates the rotational shaft 24a in a predetermined direction. Because the weight 25 is provided eccentric about the rotational shaft 24a, a vibrating motion corresponding to a rotational speed of the rotational shaft 24a occurs. The fiber vibrating device 20 itself vibrates according to the vibrating motion.

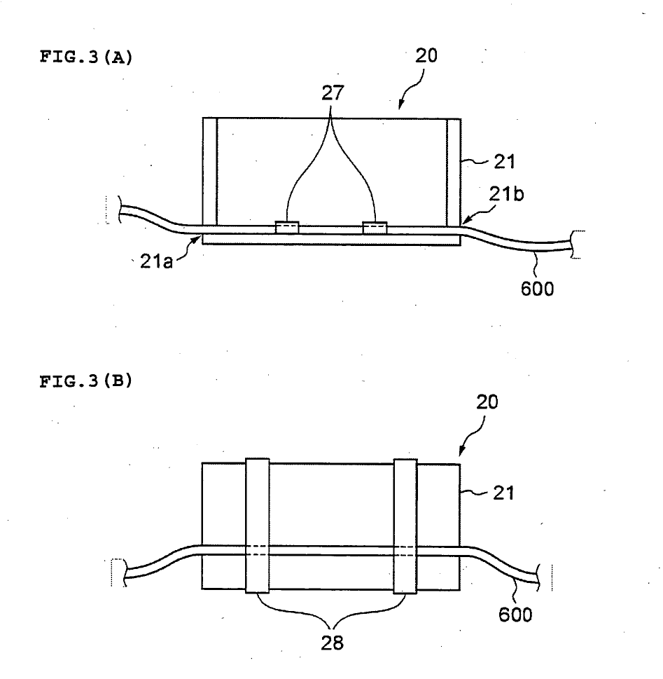

FIGS. 3A and 3B show examples of fitting configurations of the fiber vibrating device 20 to the optical fiber 600. According to the fitting configuration shown in FIG. 3A, openings 21a and 21b are formed in two opposite side surfaces of the case 21. A portion of the optical fiber 600 is inserted into the case 21 through the openings 21a and 21b. The optical fiber 600 is fixed to an inner wall of the case 21 by fixing members 27. Note that the number of fixing members 27 can be arbitrarily set.

According to the fitting configuration shown in FIG. 3B, bands 28 are provided around the case 21 of the fiber vibrating device 20 to integrally form the case 21 and the optical fiber 600 by the bands 28. Note that the number of bands 28 can be arbitrarily set. The band 28 may be a band having a ring shape in which it circuits around the case 21. The band 28 may be a band capable of fixing both ends to the case 21.

When the fiber vibrating device 20 is fit to the optical fiber 600, the vibration of the fiber vibrating device 20 itself is transferred to the optical fiber 600, thereby vibrating the optical fiber 600.

Instead of the structure in which the fiber vibrating device 20 is directly fit to the optical fiber 600 as described above, a transfer member for transferring the vibration of the fiber vibrating device 20 may be provided between the fiber vibrating device 20 and the optical fiber 600 to vibrate the optical fiber 600 through the transfer member.

[Operation]

Hereinafter, an operation of the laser operation apparatus having the above-mentioned structure according to this embodiment will be described.

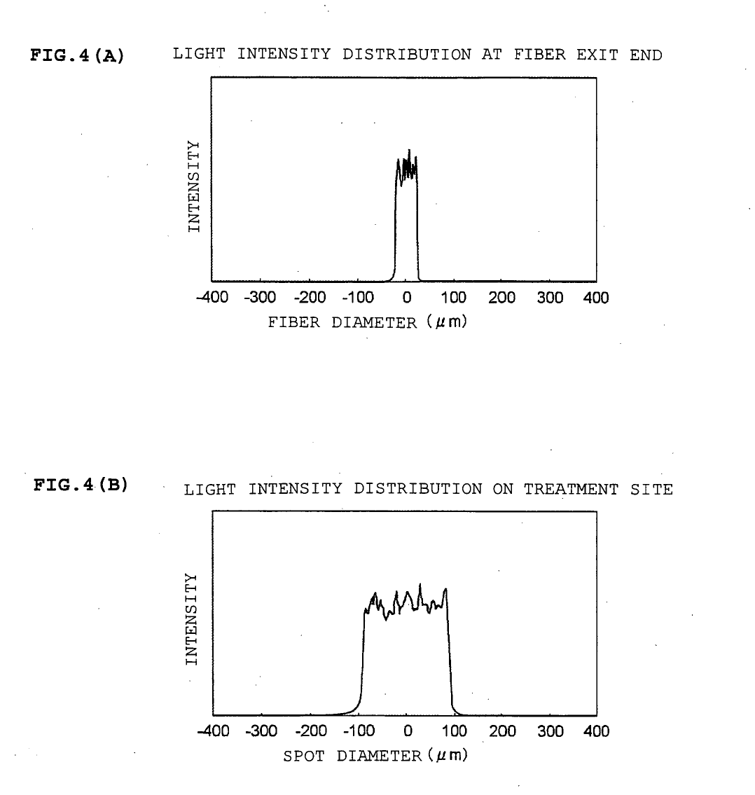

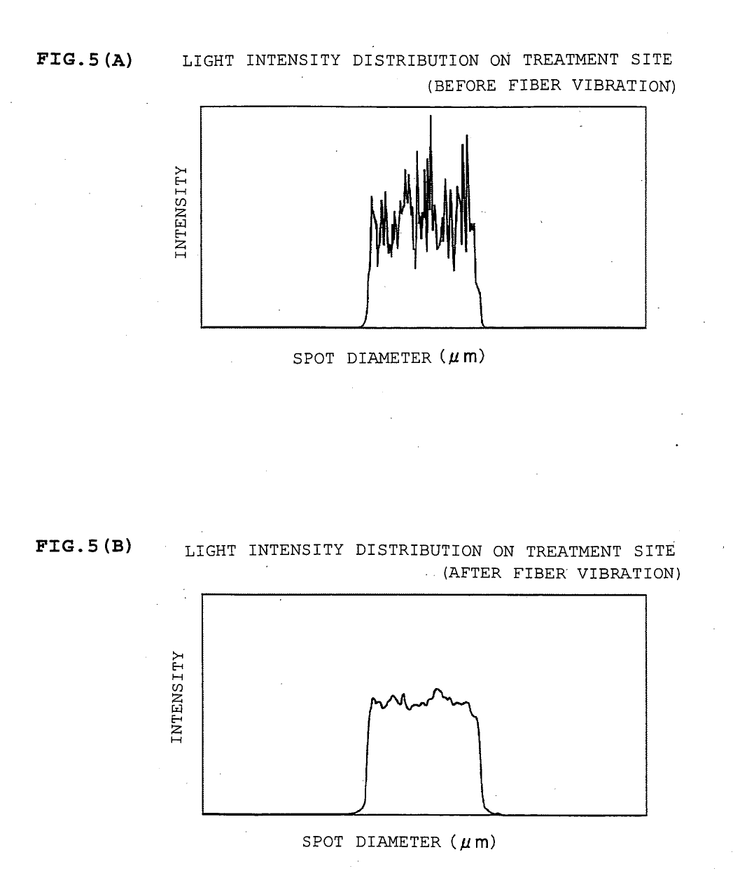

FIGS. 4(A), 4(B), 5(A), and 5(B) show measurement data for representing comparisons of the operation of the laser operation apparatus according to this embodiment and that of a conventional laser operation apparatus. FIGS. 4A and 4B show a relationship between a light intensity distribution of laser light at the exit end surface 600b of the optical fiber 600 (FIG. 4A) and a light intensity distribution of the laser light at a treatment site of the eye to be operated E (FIG. 4B). FIGS. 5A and 5B show an operation of the fiber vibrating device 20. FIG. 5A shows a light intensity distribution at the treatment site in the case where the optical fiber 600 is not vibrated. FIG. 5B shows a light intensity distribution at the treatment site in the case where the optical fiber 600 is vibrated.

First, referring to FIGS. 4A and 4B, the relationship between the light intensity distribution of the laser light at the exit end surface 600b of the optical fiber 600 and the light intensity distribution of the laser light at the treatment site will be described. Note that a fiber diameter of the optical fiber 600 is set to about 50 μm and a spot diameter of the laser light at the treatment site is set to about 200 μm.

As described above, the exit end surface 600b of the optical fiber 600 and the treatment site of the eye to be operated E are set to be optically conjugate with each other. Therefore, as shown in FIGS. 4A and 4B, the laser light at the treatment site reflects the light intensity distribution at the exit end surface 600b and becomes a substantially similar light intensity distribution according to the fiber diameter and the spot diameter. Thus, when the light intensity distribution at the treatment site is uniformed, it is apparent that the light intensity distribution at the exit end surface 600b of the optical fiber 600 may be uniformed.

Note that data shown in FIGS. 4A and 4B are obtained by a measurement using treatment laser light of a multi-mode including many modes. In the case of such a multi-mode laser light, the laser light having many modes is mixed through a process where the laser light is guided to the optical fiber 600, so that the laser light becomes a substantially rectangular light intensity distribution as shown in FIG. 4B.

On the other hand, when laser light having a single mode or a few modes is used, the laser light is not sufficiently mixed in the optical fiber 600. Therefore, as shown in FIG. 5A, the laser light becomes an extremely non-uniform light intensity distribution. The caused non-uniform light intensity distribution is generally changed according to a property (such as wavelength) of a laser light source oscillating laser light and a type of the optical fiber.

When the optical fiber 600 is vibrated by the fiber vibrating device 20 in such a case, the laser light is mixed at a vibrated portion, so that a rectangularly formed uniform light intensity distribution as shown in FIG. 5B is obtained.

As described above, even when the laser operation apparatus according to this embodiment in which the fiber vibrating device 20 is provided is the parfocal type laser operation apparatus using the step index optical fiber, it is possible to obtain the laser light having the uniformed light intensity distribution. In particular, when the light intensity distribution of the treatment laser light oscillated by the treatment laser light source 1 is uniformed, it is possible to preferably perform photo-coagulation on the treatment site of the eye to be operated E.

Note that the number of vibrations of the optical fiber 600 can be set as appropriate by controlling the rotational speed of the motor 24 or using the motor 24 having a desirable rotating rate.

In the above-mentioned embodiment, the optical fiber 600 is vibrated by the vibration caused at a time when the weight 25 provided eccentric about the rotational shaft 24a is rotated by the motor 24. However, the vibrating unit in the present invention is not limited to such a structure. For example, an actuator such as a piezoelectric element may be fit to the optical fiber 600 to vibrate the optical fiber 600 by the vibration caused by the actuator.

### VARIOUS MODIFIED EXAMPLES

Various modified examples of the laser operation apparatus according to this embodiment will be described. The modified examples described below are categorized into (1) a structure in which the operation of the fiber vibrating device 20 is associated with other operations, (2) a structure in which for controlling vibrating states (the number of vibrations and amplitude) of the optical fiber 600, and (3) a structure for preventing a vibration of the fiber vibrating device 20 and a vibrating sound thereof from transferring to the outside.

In the category (1), atypical structure is that, in particular, the start/stop of vibration of the optical fiber 600 are controlled according to a timing (start/stop) of irradiation of the laser light. The various modified examples described below are used to improve the operationality of the laser operation apparatus according to this embodiment. Note that one or plural structures of the modified examples can be applied to the laser operation apparatus according to this embodiment. It is also possible to provide a selection unit for selectively using one of plural desirable modified examples.

### Modified Example 1

### Association with Timing of Irradiation of Laser Light

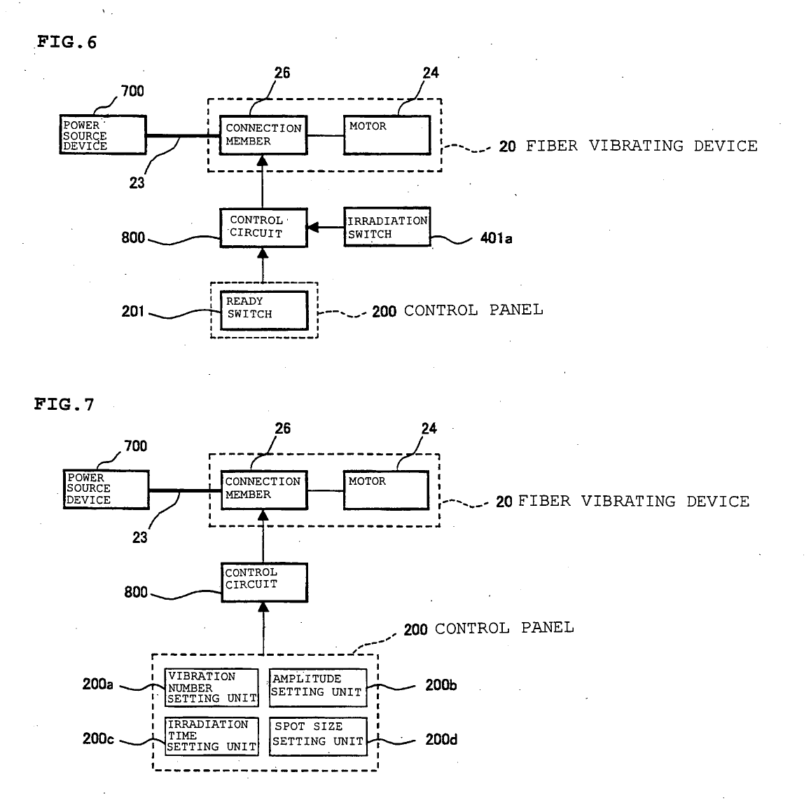

First, an example of the structure according to “association” in the category (1) will be described with reference to a block diagram shown in FIG. 6. This modified example includes a structure for operating the fiber vibrating device 20 in association with a timing of irradiation (for treatment) of the laser light.

The laser operation apparatus of the modified example 1 includes an irradiation switch 401a which is located at an upper portion of the control lever 401. When the irradiation switch 401a is pressed down, the treatment laser light is emitted from the treatment laser light source 1. Note that the foot switch 500 may be connected with the apparatus main body 100 to use the foot switch 500 as the irradiation switch.

The laser operation apparatus further includes a control circuit 800 which is composed of an arithmetic and control unit such as a CPU and memory units such as a ROM and a RAM. The control circuit 800 controls the treatment laser light source 1 which is not shown in FIG. 6 in response to an operating signal from the irradiation witch 401a to oscillate the treatment laser light.

The control circuit 800 does not enable the irradiation switch 401a until an operating signal from a ready switch 201 provided on the control panel 200 is inputted. That is, the ready switch 201 is a switch for performing laser light irradiation preparation. Therefore, unless the ready switch 201 is operated, the irradiation switch 401a is not enabled, so that the ready switch 201 acts as a safety device at a laser light irradiation.

The operation of the laser operation apparatus having the above-mentioned structure will be described. Assume that the alignment of the slit lamp 400 with the eye to be operated E is completed. First, an operator operates the ready switch 201 to obtain a state capable of emitting the treatment laser light. Then, when the irradiation switch 401a is pressed down, the control circuit 800 transmits a control signal to the connection member 26 of the fiber vibrating device 20. When the connection member 26 receives the control signal from the control circuit 800, a power supply from a power source device 700 to the motor 24 is started, so that a rotating motion of the motor 24 to which power is supplied starts. Here, because the weight 25 is provided eccentric about the rotational shaft 24a of the motor 24, the fiber vibrating device 20 starts to vibrate, thereby vibrating the optical fiber 600.

As described above, when the vibration start operation of the fiber vibrating device 20 is associated with the pressing down of the irradiation switch 401a, it is possible to control the start of vibration of the optical fiber 600 in accordance with the timing of irradiation of the treatment laser light. Therefore, the optical fiber 600 can be vibrated only when it is necessary to uniform the light intensity distribution of the laser light. Thus, the power saving of the laser operation apparatus can be realized and the generation of a vibrating sound due to the vibration of the fiber vibrating device 20 and the generation of the vibration transferred to other members can be minimized.

The control signal from the control circuit 800 may be transmitted in response to the operation of the ready switch 201. In this case, the treatment laser light can be emitted after the vibration of the optical fiber 600 which is caused by the fiber vibrating device 20 is sufficiently stabilized. Therefore, it is possible to emit the treatment laser light having a sufficiently uniformed light intensity distribution and the optical fiber 600 is not unnecessarily vibrated.

In the case of such a structure, a determination unit for determining whether or not the vibration of the optical fiber 600, that is, the vibration of the fiber vibrating device 20 is stabilized may be provided. When the determination unit determines that the vibration is stabilized, a corresponding notice is sent to an operator. The irradiation of the treatment laser light may be inhibited until the vibration is stabilized. With respect to a control example using the determination unit, for example, a time to stabilize the vibration of the optical fiber 600 (which is called a stability time) is measured in advance, an elapse time from a start of the vibration is measured by the control circuit 800 or the like. After the stability time is elapsed, the corresponding notice is sent or the inhibition of the laser light irradiation is released.

After the irradiation of the treatment laser light is completed, the vibrating motion of the fiber vibrating device 20 may be stopped. In this case, for example, when the pressing down state of the irradiation switch 401a is released, the control signal may be transmitted from the control circuit 800 to the fiber vibrating device 20 to stop the power supply to the motor 24. According to such an operation, the fiber vibrating device 20 does not continue to operate after the completion of the laser light irradiation. Thus, the power saving can be realized and the vibrating sound and the vibration transferred to other members can be minimized.

For example, a button for switching between ON/OFF of the function in the above-mentioned modified example may be provided on the control panel 200 to switch between the use and disuse of the function as appropriate (the same applies in modified examples described below).

Note that, it is also possible to employ a structure using the foot switch 500 as the ready switch according to a preference of an operator with respect to an apparatus operation, reasons with respect to an apparatus structure, and the like. In other words, the treatment laser light irradiation to be caused by the irradiation switch 401a may be disabled unless the foot switch 500 is operated. In addition, it is possible to provide a selection switch for selecting any switch to be used as the ready switch.

In the above-mentioned structure specifically described as the modified example 1, the start and end operations of vibration of the fiber vibrating device 20 are associated with the operations of the irradiation switch 401a and the ready switch 201. The modified example is not limited to this. The modified example includes a control in which the operation of the fiber vibrating device 20 is associated with an arbitrary operation related to the laser light irradiation, that is, all timings related to the laser light irradiation.

### Modified Example 2

### Association with Spot Size of Laser Light

Next, a structure for controlling the vibration of the optical fiber 600 according to a spot size of the (treatment) laser light projected to the eye to be operated E will be described with reference to a block diagram shown in FIG. 7. When the spot size of the treatment laser light is small, the unevenness of a light intensity distribution on a spot region does not become so large. Therefore, this does not hinder the treatment in many cases. On the other hand, when the spot size is large, the unevenness of a light intensity distribution on a spot region becomes considerably larger. This modified example is to avoid such a circumstance.

A laser operation apparatus of this modified example includes the control circuit 800 having the same structure as in the modified example 1 and the control panel 200 having a spot size setting unit 200d shown in FIG. 7. Although the control panel 200 shown in FIG. 7 has other setting units, it is unnecessary to provide the setting units other than the spot size setting unit 200d when this modified example is realized.

The spot size setting unit 200d provided in the control panel 200 is a setting unit for setting a spot size of laser light projected to the eye to be operated E, such as a button, a knob, or a touch panel. The operator operates the spot size setting unit 200d to set a spot size corresponding to, for example, a size of the treatment site of the eye to be operated E.

First, a threshold value S of the spot size of the laser light is set in advance. The set threshold value S is stored in, for example, the memory unit of the control circuit 800. When the spot size of the laser light can be set in a range of, for example, 50 μm to 1,000 μm, the threshold value S is set to, for example, 200 μm. The threshold value S can of course be arbitrarily set according to the properties of the fiber vibrating device 20 and the treatment laser light source 1.

In the treatment, the spot size setting unit 200d is operated to set a spot size T of the laser light. The control circuit 800 compares the set spot size T with the threshold value S. When the set spot size T is larger than the threshold value S (T>S), the control circuit 800 transmits a control signal to the fiber vibrating device 20 to control the connection member 26 so as to supply the power to the motor 24. On the other hand, when the set spot size T is smaller than or equal to the threshold value S (T≦S), the control circuit 800 does not transmit the control signal.

According to the control, when a large spot size is set, the optical fiber 600 is vibrated to uniform the light intensity distribution of the laser light. In contrast to this, when the spot size is small and it is unnecessary to uniform the light intensity distribution, the optical fiber 600 is not vibrated. Therefore, the ON/OFF of the uniformity of the light intensity distribution can be automatically switched depending on the necessity, so that the same effect as in the modified example 1, such as power saving can be obtained.

### Modified Example 3

### Association with Irradiation Time of Laser Light

Next, a structure for controlling the vibration of the optical fiber 600 according to an irradiation time of the treatment laser light will be described with reference to FIG. 7.

The control panel 200 in a laser operation apparatus of this modified example includes an irradiation time setting unit 200c for setting the irradiation time of the treatment laser light. The irradiation time setting unit 200c is a setting unit such as a button, a knob, or a touch panel.

When the irradiation time setting unit 200c is operated to set the irradiation time of the treatment laser light, the control circuit 800 sets a time for which the power is supplied to the motor 24 (which is called a power supply time). The power supply time may be substantially equal to the irradiation time. When the irradiation switch 401a is pressed down after the operation of the ready switch 201, the control circuit 800 simultaneously starts a time measurement and an irradiation of the treatment laser light. Further, the control circuit 800 transmits a control signal to the fiber vibrating device 20 to supply the power to the motor 24, thereby vibrating the optical fiber 600. When the irradiation time elapses, the irradiation of the treatment laser light is completed. When the power supply time elapses, a control signal is transmitted to the fiber vibrating device 20 to stop the power supply to the motor 24, thereby stopping the vibration of the optical fiber 600.

According to the control in this modified example, the optical fiber 600 can be vibrated according to the irradiation time of the treatment laser light. Therefore, there is no case where the fiber vibrating device 20 is wastefully operated, so that the same effect as in the modified example 1, such as power saving can be obtained.

Instead of setting the power supply time according to the irradiation time set by the irradiation time setting unit 200c, the number of vibrations of the fiber vibrating device 20 (that is, the number of revolutions of the motor 24) may be set to vibrate the fiber vibrating-device 20, that is, the optical fiber 600 by the number of vibrations.

### Modified Example 4

### Control of the Number of Vibrations of Optical Fiber

A modified example according to “control of vibrating states” in the category (2) will be described. A laser operation apparatus of the modified example 4 has a structure for controlling the number of vibrations of the optical fiber 600. More specifically, in this modified example, the control panel 200 includes a vibration number setting unit 200a as shown in FIG. 7. The vibration number setting unit 200a is a setting unit such as a button, a knob, or a touch panel.

When an operator or the like operates the vibration number setting unit 200a to set the number of vibrations (for example, N) of the optical fiber 600, a set signal is transmitted from the control panel 200 to the control circuit 800. The control circuit 800 produces a control signal based on the set signal and transmits the control signal to the fiber vibrating device 20.

At this time, for example, the control signal is produced as follows. First, the number of vibrations f (Hz) of the fiber vibrating device 20 based on, for example, a rotational speed of the motor 24 is measured in advance and stored in the memory unit of the control circuit 800. When the set signal from the control panel 200 is received to recognize the set number of vibrations N, the control circuit 800 divides the set number of vibrations N by the number of vibrations f to obtain a vibration time t=N/f (sec). Then, the control circuit 800 transmits a control signal for starting the vibration to the fiber vibrating device 20 and simultaneously starts the time measurement. When the measured time reaches the vibration time t (sec), a control signal for stopping the vibration is transmitted to the fiber vibrating device 20 to stop the vibration of the fiber vibrating device 20, that is, the vibration of the optical fiber 600.

The control signal for starting the vibration is a signal for controlling the connection member 26 so as to start the power supply to the motor 24 and the control signal for stopping the vibration is a signal for controlling the connection member 26 so as to stop the power supply to the motor 24.

Note that, when the laser operation apparatus includes a changing unit for changing the number of vibrations of the fiber vibrating device 20 (the number of vibrations of the optical fiber 600) by changing the rotational speed of the motor 24, the control circuit 800 computes the vibration time according to the number of vibrations of the optical fiber 600 which is set by the changing unit.

According to the control for the number of vibrations of the optical fiber 600 in the laser operation apparatus of this modified example, the number of vibrations of the optical fiber 600 can be set as appropriate based on a treatment time (such as a treatment laser light irradiation time). A structure for realizing the set number of vibrations is not limited to the above-mentioned example. For example, a unit for counting the vibration of the fiber vibrating device 20 may be provided to control the number of vibrations of the optical fiber 600 based on a count result.

### Modified Example 5

### Control of Amplitude of Optical Fiber

Next, a modified example including a structure for controlling an amplitude of the optical fiber 600 vibrated by the fiber vibrating device 20 will be described. The control panel 200 in laser operation apparatus of this modified example includes an amplitude setting unit 200b for setting an amplitude of the optical fiber 600, that is, an amplitude of the fiber vibrating device 20. The amplitude setting unit 200b is a setting unit such as a button, a knob, or a touch panel.

Although not shown, the fiber vibrating device 20 further includes a plurality of motors. A weight member having a different weight and a different degree of eccentricity is provided to the rotational shaft of each of the motors. Each of the motors receives the power from the power source through the connection member 26. The connection member 26 is constructed to selectively supply the power to one of the respective motors.

When an operator or the like operates the amplitude setting unit 200b to set a desirable amplitude, a set signal is transmitted from the control panel 200 to the control circuit 800. When the set signal is received, the control circuit 800 produces a control signal for selecting any motor of the plurality of motors, to which the power is supplied and transmits the control signal to the connection member 26 of the fiber vibrating apparatus 20. The connection member 26 selects a power supply destination (motor) based on the control signal to rotate the selected motor, thereby vibrating the fiber vibrating device 20 at the set amplitude. According to such an amplitude control, a suitable amplitude can be realized. Therefore, there is no case where the vibration becomes larger for nothing, so that the vibrating sound due to the increased vibration and the vibration transferred to other members can be minimized.

In the above-mentioned structure, the weight member having a different weight and a different degree of eccentricity is provided to each of the plurality of motors and one of the motors is selectively operated to control the amplitude. However, this modified example is not limited to the structure. For example, a plurality of weight members are arranged to be selectively attachable to the rotational shaft of a motor. Then, one of the plurality of weight members may be attached to the rotational shaft according to a set operation of the amplitude setting unit 200b to rotate the motor. Alternatively, an attachment position of a weight member to the rotational shaft of a motor is made changeable. Then, the attachment position of the weight member is changed according to a set operation of the amplitude setting unit 200b to change the degree of eccentricity of the weight member, so that the amplitude can be controlled.

When the fiber vibrating device 20 to which the optical fiber 600 is fit is placed on the table 300 or the like, the amplitude of the optical fiber 600 can be controlled by the following structure. Although not shown, a housing case for housing the fiber vibrating device 20 is provided. A distance between at least one pair of opposite side surfaces of the housing case is made changeable. That is, a drive unit such as a motor for actuating the one pair of opposite side surfaces in fitting and dividing directions is provided in the housing case. The drive unit is controlled by the control circuit 800. The fiber vibrating device 20 is housed in the housing case so as to vibrate the fiber vibrating device 20 in a direction parallel to the one pair of opposite side surfaces and placed on the table 300 or the like.

When the operator or the like operates the amplitude setting unit 200b to set a desirable amplitude, a set signal is transmitted to the control circuit 800. The control circuit 800 produces a control signal for controlling the drive unit of the housing case based on the set signal and transmits the control signal to the drive unit of the housing case. The drive unit actuates the one pair of opposite side surfaces in the fitting and dividing directions based on the control signal from the control circuit 800.

When the fiber vibrating device 20 is vibrated with this state, the vibration direction coincides with the direction parallel to the one pair of opposite side surfaces of the housing case. Therefore, a half of a length obtained by dividing the distance between the one pair of opposite side surfaces by the width of the case 21 of the fiber vibrating device 20 in the vibration direction becomes the amplitude of the fiber vibrating device 20, that is, the amplitude of the optical fiber 600. According to the amplitude control, it is unnecessary to increase the weight of the fiber vibrating device 20 and the amplitude can be successively changed. A structure for realizing this is simple.

### Modified Example 6

### Preventions of Vibration of Fiber Vibrating Device and Vibrating Sound thereof from Transferring to Outside

A laser operation apparatus of a modified example according to this embodiment, which is included in the category (3) will be described.

As an example, in order to prevent the vibrating sound of the fiber vibrating device 20 from transferring, a structure in which a soundproof member having a soundproof function is bonded to the inner wall of the case 21 of the fiber vibrating device 20 can be employed. In order to reduce a rotating sound of the motor 24 itself, a silent motor can be used or a soundproof member (soundproof unit in the present invention) can be provided around the motor 24.

When the housing case described in the modified example 5 is used, a member having a soundproof function or a vibration-proof function may be provided on the inner wall or the outer wall of the housing case. A vibration-proof member which is an elastic member such as a rubber or a spring (vibration-proof unit in the present invention) may be located between the housing case and the table 300 on which the housing case is placed, so that the vibration of the fiber vibrating device 20 is not transferred to the table 300 and the like.

As described above, the fiber vibrating device 20 may be provided at any position of the optical fiber 600 as long as the incident end surface 600a and the exit end surface 600b of the optical fiber 600 are not displaced. For example, the fiber vibrating device 20 can be located in the apparatus main body 100 or at a connector portion 600c connecting with the apparatus main body 100, which is formed on the incident end surface 600a side of the optical fiber 600. In this case, the soundproof member or the vibration-proof member can be provided on the inner wall of the case of the apparatus main body 100 or on the inner wall of the connector portion 600c.

When the soundproof function or the vibration-proof function is provided, there is no case where the operation is hindered and a patient is made uncomfortable. In particular, it is possible to avoid a fear that an alignment state of the slit lamp 400 with the eye to be operated E is disturbed by the vibration.

### ANOTHER MODIFIED EXAMPLE

Next, another modified example of the laser operation apparatus according to this embodiment will be described. According to the structures described-above, the optical fiber 600 for guiding the laser light from the apparatus main body 100 to the slit lamp 400 is vibrated. In the laser operation apparatus of this modified example, the fiber vibrating device 20 is included in the apparatus main body 100.

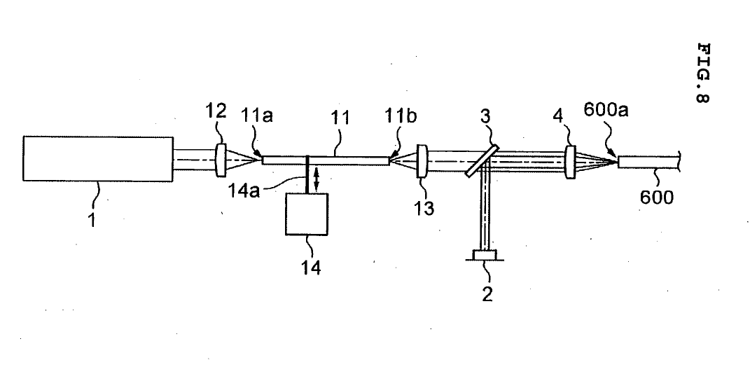

FIG. 8 shows a part of the structure housed in the apparatus main body 100 (the same reference numeral is used) of the laser operation apparatus of this modified example. The apparatus main body 100 includes, in addition to the treatment laser light source 1, the sighting laser light source 2, the dichroic mirror 3, and the condensing lens 4, an optical fiber 11, a condensing lens 12 for condensing treatment laser light from the treatment laser light source 1 to an incident end surface 11a of the optical fiber 11, a collimator lens for converting the treatment laser light exited from an exit end surface 11b of the optical fiber 11 into a parallel light flux, and a fiber vibrating device 14 for providing a vibration to the optical fiber 11.

A connection portion 14a whose end is connected with the optical fiber 11 and that transfers the vibration to the optical fiber 11 is provided in the fiber vibrating device 14. The fiber vibrating device 14 is composed of a solenoid for producing a linear reciprocating motion, or the like. Note that the fiber vibrating device 20 according to the above-mentioned embodiment maybe directly fit to the optical fiber 11 to vibrate the optical fiber 11.

According to this modified example, it is possible to emit the treatment laser light having the uniformed light intensity distribution. It is unnecessary to uniform the light intensity distribution of the sighting laser light, so that the structure of this modified example is sufficient in practical use. Because the fiber vibrating device 14 is housed in the inner portion of the apparatus main body 100, there is an advantageous in that a structure for soundproof and vibration-proof is easy to provide. As compared with the conventional case using the diffusion plate, a loss of laser light is small.

In this modified example, in addition to the optical fiber 600 joining the main body 100 to the slip lamp 400, the optical fiber 11 is provided in the inner portion of the apparatus main body 100, so that a loss of laser light becomes larger as compared with the laser operation apparatus according to the above-mentioned embodiment. Therefore, it is preferable to employ the structure of this modified example in the case where the treatment laser light source 1 has a sufficient margin of laser light output.

The above-mentioned structures for various controls can be added as appropriate to the laser operation apparatus of this modified example. Both the fiber vibrating device 20 and fiber vibrating device 14 may be provided to further improve the uniformity of the light intensity distribution of the laser light. In this case, it is possible to separately control the fiber vibrating device 20 and fiber vibrating device 14.

Plural portions of the optical fiber 600 may be vibrated. Similarly, plural portions of the optical fiber 11 may be vibrated. In order to realize this, a plurality of fiber vibrating devices 20 (14) may be provided or the plural portions of the optical fiber 600 (11) may be vibrated by the single fiber vibrating device 20 (14). When the plural portions of the optical fiber 600 (11) are vibrated, the vibration directions of the respective portions may be substantially idential to one another or different from one another (which are set to, for example, two directions orthogonal to each other).

In the laser operation apparatus according to the present invention, the structure for uniforming the light intensity distribution of the (for treatment) laser light can be provided at an arbitrary position between the treatment laser light source 1 and the eye to be operated E. For example, such a structure may be located in the slip lamp 400.

A photo-coagulation apparatus is described as the laser operation apparatus of the present invention. As long as the laser operation apparatus is an apparatus for performing a treatment (operation) by irradiating a treatment site of a patient, in particular, an ophthalmologic treatment site with laser light, any kind of apparatus can be applicable. For example, the structure of the present invention can be applied to a laser light irradiating probe used with an ophthalmologic operation microscope. Here, when the probe itself is vibrated to uniform the light intensity distribution of the laser light, the safety is reduced. Therefore, it is preferable to employ, for example, a structure for vibrating the optical fiber guiding the laser light to the probe.

The structures described above are examples of the laser operation apparatus according to the present invention. Therefore, modifications, alternations, additions, and the like of the structures can be arbitrarily made if necessary without departing from a scope of the present invention. The structures according to the present invention can be applied to not only the laser operation apparatus for performing the photo-coagulation on the eye to be operated but also, for example, a laser operation apparatus for performing a photo dynamic therapy (PDT).

According to the first aspect of the present invention, the optical fiber guiding the laser light is vibrated, so that the light intensity distribution of the laser light can be easily and effectively uniformed.

According to the second aspect of the present invention, the start/stop of the vibration of the optical fiber which is caused by the vibrating means are controlled in association with the timing of irradiation of the laser light, so that the operationality of the apparatus is improved.

According to the third aspect of the present invention, the vibration of the optical fiber which is caused by the vibrating means is started in association with the operation of the irradiation switch, that is, the starting of irradiation of the laser light, so that the operationality of the apparatus is improved.

According to the fourth aspect of the present invention, the vibration of the optical fiber which is caused by the vibrating means is started in association with the operation of the ready switch, so that the operationality of the apparatus is improved. In addition, the vibration of the optical fiber is stabilized before the time of irradiation of the laser light, so that the laser light having the sufficiently uniformed light intensity distribution can be emitted.

1. A laser operation apparatus, comprising:
a laser oscillator;
an optical fiber for guiding laser light oscillated by the laser oscillator;
an irradiation optical system for irradiating a treatment site of a patient with the laser light guided through the optical fiber during an alignment with the treatment site; and
vibrating device for vibrating a portion of the optical fiber in a direction substantially orthogonal to a longitudinal direction of the optical fiber.

2. A laser operation apparatus according to claim 1, wherein the vibrating device is controlled so as to start and stop a vibration of the optical fiber in accordance with a timing of irradiation of the treatment site with the laser light from the irradiation optical system.

3. A laser operation apparatus according to claim 2, further comprising an irradiation switch operated to start the irradiation of the treatment site with the laser light from the irradiation optical system,
wherein the vibrating device is controlled so as to start the vibration of the optical fiber in accordance with an operation of the irradiation switch.

4. A laser operation apparatus according to claim 3, further comprising a ready switch operated before the operation of the irradiation switch in order to enable the irradiation switch,
wherein the vibrating device is controlled so as to start the vibration of the optical fiber in accordance with an operation of the ready switch.
